(19) United States
(12) Patent Application Publication
(10) Pub. No.: US 2022/0403435 A1
(43) Pub. Date: Dec. 22, 2022

(54) Increasing Long-Sequence Yields In Template-Free Enzymatic Synthesis of Polynucleotides

(71) Applicant: DNA Script SAS, Le Kremlin-Bicêtre (FR)

(72) Inventors: Adrian Horgan, Le Kremlin-Bicêtre (FR); Ivo Sarac, Le Kremlin-Bicêtre (FR); Supaporn Niyomchon, Le Kremlin-Bicêtre (FR); Xavier Godron, Le Kremlin-Bicêtre (FR)

(21) Appl. No.: 17/629,322

(22) PCT Filed: Jul. 28, 2020
(86) PCT No.: PCT/EP2020/071316

§ 371 Date: Jan. 21, 2022

(30) Foreign Application Priority Data
Aug. 1, 2019 (EP) 19189639.8
Oct. 1, 2019 (EP) 19200740.9

(51) Int. Cl. C12P 19/34; C12N 9/12

(52) U.S. Cl. CPC C12P 19/34; C12N 9/1264; C12Y 207/07031

(57) ABSTRACT

The present invention is directed to methods and kits for template-free enzymatic synthesis of polynucleotides using chain elongation conditions that suppress the formation of DNA secondary structures including, but not limited to, intra-strand and between-strand duplexes, G-quadruplexes, and the like. In some embodiments, such chain elongation conditions include using 3′-O-blocked dNTP monomers that base protection groups or base analogs that suppress the formation of hydrogen bonding in the polynucleotide being synthesized.

### CROSS-REFERENCE TO RELATED APPLICATIONS

This application is a 371 of International Application Serial No. PCT/EP2020/071316, filed on Jul. 28, 2020, which application claims priority to EP19189639.8, filed on Aug. 1, 2019, and EP19200740.9, filed on Oct. 1, 2019, the disclosures of which are incorporated herein by reference in their entirety.

### INCORPORATION BY REFERENCE OF SEQUENCE LISTING PROVIDED AS A TEXT FILE

A Sequence Listing is provided herewith in a text file “DNAS-011_SEQ_LIST_ RevJun2022_ST25” created on Jun. 2, 2022 and having a size of 105,441 bytes. The contents of the text file are incorporated herein by reference in their entirety.

### BACKGROUND

Interest in enzymatic approaches to polynucleotide synthesis has recently increased not only because of increased demand for synthetic polynucleotides in many areas, such as synthetic biology, CRISPR-Cas9 applications, and high-throughput sequencing, but also because of the limitations of chemical approaches to polynucleotide synthesis, such as upper limits on product length and the use and needed disposal of organic solvents, Jensen et al, Biochemistry, 57: 1821-1832 (2018). Enzymatic synthesis is attractive because its specificity and efficiency and its requirement of mild aqueous reaction conditions.

Currently, most enzymatic approaches employ a template-free polymerase to repeatedly add 3′-O-blocked nucleoside triphosphates to a single stranded initiator or an elongated strand attached to a support followed by deblocking until a polynucleotide of the desired sequence is obtained. An objective of such methods is to provide long synthetic nucleic acids whose structures are indistinguishable from natural counterparts. However, as polynucleotide length increases the possibility of secondary structures forming, such as, for example, intra-strand or cross-strand duplexes, increases, with the consequence that synthesis reagents lose access to reaction sites and product yields drop.

In view of the above, template-free enzymatic synthesis of polynucleotides would be advanced if methods were available to reduce the formation of secondary structures that reduce the yield of the desired polynucleotide product.

### SUMMARY OF THE INVENTION

The present invention is directed to methods for template-free enzymatic synthesis of polynucleotides that employ base analogs and base protecting moieties for the purpose of reducing the formation of secondary structures during synthesis. In one aspect, such methods employ base protecting moieties attached to exocyclic amine of adenine, cytosine and guanine. In another aspect, such base protecting moieties may also include moieties providing additional functionalities, such as, capture moieties, nuclease blockers, reporters, or the like. For example, capture moieties, such as biotin, which may be employed to capture polynucleotide after synthesis prior to deprotection.

In some embodiments, the invention is directed to methods of synthesizing a polynucleotide having a predetermined sequence comprising the steps of: a) providing an initiator having a free 3′-hydroxyl; b) repeating until the polynucleotide is complete cycles of (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′- hydroxyls, until the polynucleotide is formed, wherein the elongation conditions are selected to prevent hydrogen bonding or base stacking. In some embodiments, conditions selected to prevent intra-molecular or cross-molecular hydrogen bonding include providing 3′-O-blocked nucleoside triphosphate monomers with base protecting moieties that preclude the protected groups from participating in hydrogen bonding. In particular, said elongation conditions may provide that at least one 3′-O-blocked nucleoside triphosphate has a base protecting moiety attached to its base to prevent hydrogen bonding, preferably to a nitrogen or to an oxygen of its base, more preferably to a nitrogen. Said nitrogen of said base of said 3′-O-blocked nucleoside triphosphate may be an exocyclic nitrogen. In some particular embodiments, said base protecting moiety may be attached to 6-nitrogen of deoxyadenosine triphosphate, 2-nitrogen of deoxyguanosine triphosphate, or 4-nitrogen of deoxycytidine triphosphate. Said base protecting moiety may be an acyl protecting group. In particular, said base protecting moiety attached to said 6-nitrogen of deoxyadeno sine triphosphate may be selected from the group consisting of benzoyl, phthaloyl, phenoxyacetyl, and methoxyacetyl; said base protecting moiety attached to said 2-nitrogen of deoxyguanosine triphosphate may be selected from the group consisting of isobutyryl, isobutyryloxyethylene, acetyl, 4-isopropyl-phenoxyacetyl, phenoxyacetyl, and methoxyacetyl; and said base protecting moiety attached to said 4-nitrogen of deoxycytidine triphosphate may be selected from the group consisting of benzoyl, phthaloyl, acetyl, and isobutyryl. Alternatively, said base protecting moiety attached to said 6-nitrogen of deoxyadenosine triphosphate may be benzoyl or dimethylformamidine, preferably dimethylformamidine; said base protecting moiety attached to said 2-nitrogen of deoxyguanosine triphosphate may be acetyl or dimethylformamidine, preferably dimethylformamidine; and said base protecting moiety attached to said 4-nitrogen of deoxycytidine triphosphate may be acetyl. In some embodiments, the base protecting moiety may be base labile, in particular may be amidine. The method may include removing said base protecting moieties from nucleotides of the polynucleotide. The initiator may be attached to a solid support. In some particular embodiments, said initiator comprises a base-cleavable nucleoside and said base protecting moieties are base labile and said step of removing comprises treating said polynucleotide with base so that base protecting moieties and the base-cleavable nucleoside are cleaved in the same reaction. In some embodiments, conditions selected to prevent intra-molecular or cross-molecular hydrogen bonding may include the presence of denaturation agents, preferably selected from the group consisting of water miscible solvents having a dielectic constant less than that of water and chaotropic agents, more particularly selected from the group consisting of formamide, guanidine, sodium salicylate, dimethyl sulfoxide (DMSO), propylene glycol, and urea. Preferably, said 3′-O-protecting group may be selected from the group consisting of 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl. More preferably, said 3′-O-protecting group is azidomethyl or amine. When base-protecting moieties are employed, after synthesis is complete, methods of the invention may include a further step of removing the base protecting moiety from nucleotides of the final product.

In some embodiments, the invention is directed to methods of synthesizing a polynucleotide having a predetermined sequence, comprising the steps of: a) providing an initiator having a free 3′-hydroxyl; b) repeating until the polynucleotide is synthesized cycles of (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′-hydroxyls, until the polynucleotide is formed, wherein the elongation conditions are selected to prevent hydrogen bonding or base stacking; wherein a final cycle comprises only step (i) and wherein the 3′-O-blocked, base protected nucleoside triphosphate comprises a base protecting moiety comprising a capture moiety; and c) capturing the polynucleotide with a complement of the capture moiety. Said method may further comprise a step of deblocking said captured polynucleotide.

### BRIEF DESCRIPTION OF THE DRAWINGS

FIG. 1A diagrammatically illustrates a method of template-free enzymatic synthesis of a polynucleotide.

FIG. 1B diagrammatically illustrates types of secondary structures that may form which inhibit synthesis reagent access to growing chains.

FIG. 2 shows a scheme for synthesizing a class of base-protected 3′-O-amino-2′-deoxynucleoside triphosphates.

FIG. 3 shows data illustrating increased yields of (dG)io when synthesized using monomers comprising 3′-O-NH2-N2-acecyl-2′-deoxyguanosine triphosphate.

FIGS. 4A-4B illustrate exemplary base protecting moieties that include moieties with additional functionalities, such as, capture moieties.

### DETAILED DESCRIPTION OF THE INVENTION

The general principles of the invention are disclosed in more detail herein particularly by way of examples, such as those shown in the drawings and described in detail. It should be understood, however, that the intention is not to limit the invention to the particular embodiments described. The invention is amenable to various modifications and alternative forms, specifics of which are shown for several embodiments. The intention is to cover all modifications, equivalents, and alternatives falling within the principles and scope of the invention.

The practice of the present invention may employ, unless otherwise indicated, conventional techniques and descriptions of organic chemistry, molecular biology (including recombinant techniques), cell biology, and biochemistry, which are within the skill of the art. Such conventional techniques may include, but are not limited to, preparation and use of synthetic peptides, synthetic polynucleotides, monoclonal antibodies, nucleic acid cloning, amplification, sequencing and analysis, and related techniques. Protocols for such conventional techniques can be found in product literature from manufacturers and in standard laboratory manuals, such as Genome Analysis: A Laboratory Manual Series (Vols. I-IV); PCR Primer: A Laboratory Manual; and Molecular Cloning: A Laboratory Manual (all from Cold Spring Harbor Laboratory Press); Lutz and Bornscheuer, Editors, Protein Engineering Handbook (Wiley-VCH, 2009); Hermanson, Bioconjugate Techniques, Second Edition (Academic Press, 2008); and like references.

The invention is directed to improvements to template-free enzymatic synthesis of polynucleotides, especially DNA, which permit higher yields of long polynucleotides by providing synthesis conditions that suppress the formation of secondary structures in growing chains, such as, caused by hydrogen bonding, base stacking, and the like. Without the intention of being limited to a particular theory or hypothesis, it is believed that the formation of such secondary structures limit access to synthesis reagents, such as template-free polymerases, thereby inhibiting chain extension and increasing the variability of product length. In part, the invention is based on a recognition and appreciation that the negative effects of such secondary structures on product yield may be mitigated or suppressed by selecting elongation conditions that include higher reaction temperature, e.g. by using thermal stable template-free polymerases; presence of denaturation agents; and use of monomers that have base analogs or base protecting moieties attached to groups, such as exocyclic amines, to prevent hydrogen bonding.

In some embodiments, the invention includes the use of base-protecting moieties that not only prevent formation of secondary structures, e.g. by preventing hydrogen bonding, but also provide an additional functionality such as moieties that block exonuclease activity, serve as reporter groups, serve as capture moieties, or the like. For example, a base-protecting moiety may comprise a molecular capture moiety that permits facile isolation of polynucleotide products which, in turn, may be released by deprotection without leaving unnatural adducts or “scarring” on the product.

### Template-Free Enzymatic Synthesis

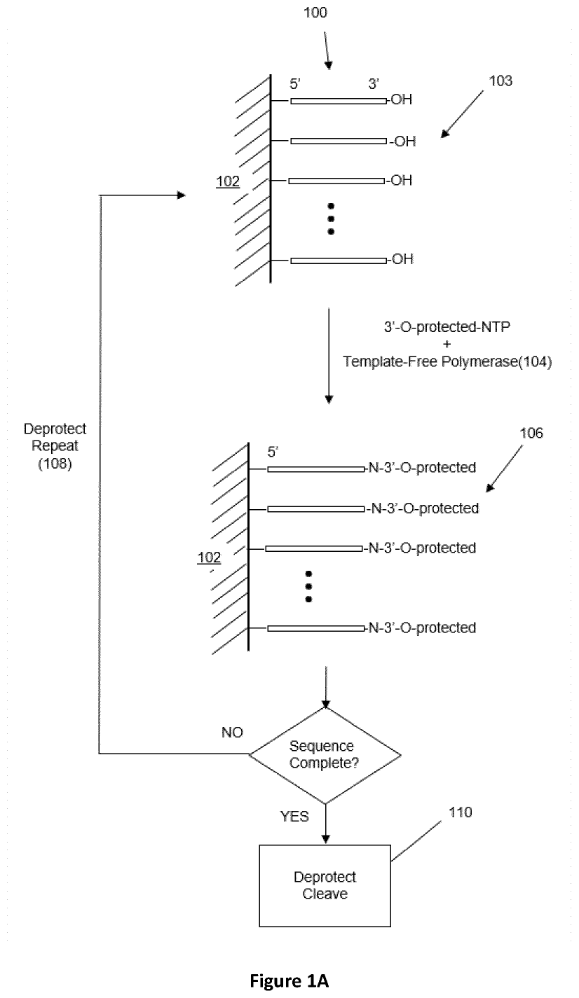

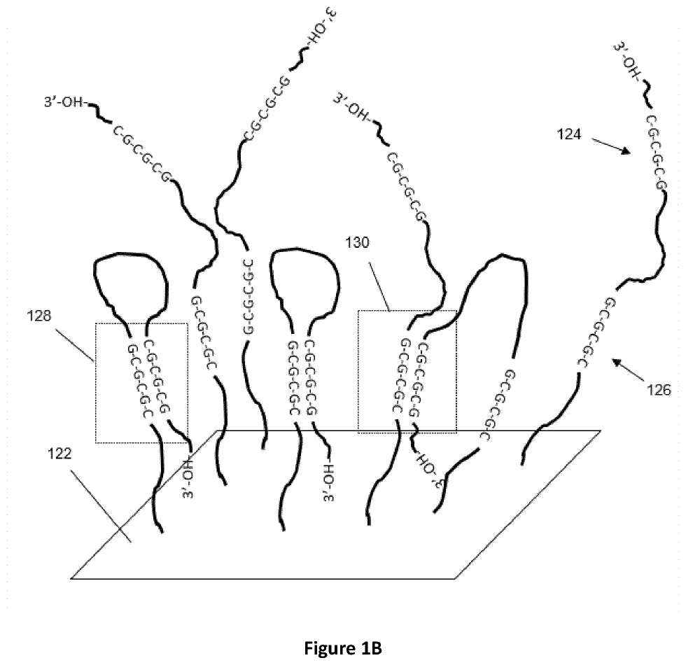

Generally, methods of template-free (or equivalently, “template-independent”) enzymatic DNA synthesis comprise repeated cycles of steps, such as are illustrated in FIG. 1A, in which a predetermined nucleotide is coupled to an initiator or growing chain in each cycle. The general elements of template-free enzymatic synthesis is described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5763594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

Initiator polynucleotides (100) are provided, for example, attached to solid support (102), which have free 3′-hydroxyl groups (103). To the initiator polynucleotides (100) (or elongated initiator polynucleotides in subsequent cycles) are added a 3′-O-protected-dNTP and a template-free polymerase, such as a TdT or variant thereof (e.g. Ybert et al, WO/2017/216472; Champion et al, WO2019/135007) under conditions (104) effective for the enzymatic incorporation of the 3′-O-protected-dNTP onto the 3′ end of the initiator polynucleotides (100) (or elongated initiator polynucleotides). (The terms “protected” and “blocked,” and their cognates, in reference to groups on nucleotide monomers are used interchangeably and synonymously.) This reaction produces elongated initiator polynucleotides whose 3′-hydroxyls are protected (106). If the elongated initiator polynucleotide contains a competed sequence, then the 3′-O-protection group may be removed, or deprotected, and the desired sequence may be cleaved from the original initiator polynucleotide. Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, an “initiator” (or equivalent terms, such as, “initiating fragment,” “initiator nucleic acid,” “initiator oligonucleotide,” or the like) usually refers to a short oligonucleotide sequence with a free 3′-end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides. In some embodiments, the initiating fragment is single-stranded. In alternative embodiments, the initiating fragment is double-stranded. In some embodiments, an initiator may comprise a non-nucleic acid compound having a free hydroxyl to which a TdT may couple a 3′-O-protected dNTP, e.g. Baiga, U.S. Pat. publications US2019/0078065 and US2019/0078126.

After synthesis is completed polynucleotides with the desired nucleotide sequence may be released from initiators and the solid supports by cleavage. A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide. Further methods for cleaving single stranded polynucleotides are disclosed in the following references, which are incorporated by reference: U.S. Pat. Nos. 5,739,386, 5,700,642 and 5,830,655; and U.S. Pat. Publication Nos. 2003/0186226 and 2004/0106728; and in Urdea and Horn, U.S. Pat. No. 5,367,066.

In some embodiments, cleavage by glycosylases and/or endonucleases may require a double stranded DNA substrate.

Returning to FIG. 1A, in some embodiments, an ordered sequence of nucleotides are coupled to an initiator nucleic acid using a template-free polymerase, such as TdT, in the presence of 3′-O-protected dNTPs in each synthesis step. In some embodiments, the method of synthesizing an oligonucleotide comprises the steps of (a) providing an initiator having a free 3′-hydroxyl; (b) reacting under extension conditions the initiator or an extension intermediate having a free 3′-hydroxyl with a template-free polymerase in the presence of a 3′-O-protected nucleoside triphosphate to produce a 3′-O-protected extension intermediate; (c) deprotecting the extension intermediate to produce an extension intermediate with a free 3′-hydroxyl; and (d) repeating steps (b) and (c) until the polynucleotide is synthesized. (Sometimes the terms “extension intermediate” and “elongation fragment” are used interchangeably and synonymously). In some embodiments, an initiator is provided as an oligonucleotide attached to a solid support, e.g. by its 5′ end. The above method may also include washing steps after the reaction, or extension, step, as well as after the de-protecting step. For example, the step of reacting may include a sub-step of removing unincorporated nucleoside triphosphates, e.g. by washing, after a predetermined incubation period, or reaction time. Such predetermined incubation periods or reaction times may be a few seconds, e.g. 30 sec, to several minutes, e.g. 30 min.

As illustrated in FIG. 1B, when the sequence of polynucleotides on a synthesis support (122) include reverse complementary subsequences, e.g. (124) and (126), secondary intra-molecular (128) or cross-molecular (130) structures may be created by the formation of hydrogen bonds between the reverse complementary regions. In one aspect of the invention, base protecting moieties for exocyclic amines are selected so that hydrogens of the protected nitrogen cannot participate in hydrogen bonding, thereby preventing the formation of secondary structures, such as those illustrated in FIG. 1B. That is, in one aspect of the invention, base protecting moieties are selected to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

3′-O-blocked dNTPs without base protection may be purchased from commercial vendors or synthesized using published techniques, e.g. U.S. Pat. No. 7,057,026; Guo et al, Proc. Natl. Acad. Sci., 105(27): 9145-9150 (2008); Benner, U.S. Pat. Nos. 7,544,794 and 8,212,020; International patent publications WO2004/005667, WO91/06678; Canard et al, Gene (cited herein); Metzker et al, Nucleic Acids Research, 22: 4259-4267 (1994); Meng et al, J. Org. Chem., 14: 3248-3252 (3006); U.S. Pat. publication 2005/037991. 3′-O-blocked dNTPs with base protection may be synthesized as described below.

When base-protected dNTPs are employed the above method of Fig. 1A may further include a step (e) removing base protecting moieties, which in the case of acyl or amidine protection groups may (for example) include treating with concentrated ammonia.

The above method may also include capping step(s) as well as washing steps after the reacting, or extending, step, as well as after the deprotecting step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions.

In some embodiments, reaction conditions for an extension or elongation step may comprising the following: 2.0 μM purified TdT; 125-600 μM 3′-O-blocked dNTP (e.g. 3′-O-NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3 minutes. In embodiments, in which the 3′-O-blocked dNTPs are 3′-O-NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out in a 50 μL volume, at a temperature within the range of RT to 45° C. for 30 seconds to several minutes.

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. Pat. Nos. 7,544,794 and 8,212,020; U.S. Pat. Nos. 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments of the invention.

| | |
|---|---|
| 3′-O—NH2 | 3′-O-azidomethyl |
| 3′-O—NH2 | 3′-O-allyl, 3′-O-propargyl |
| 3′-O—NH2 | 3′-O-phosphate |
| 3′-O-azidomethyl | 3′-O-allyl, 3′-O-propargyl |
| 3′-O-azidomethyl | 3′-O-phosphate |
| 3′-O-allyl, 3′-O-propargyl | 3′-O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C.

A “3′-phosphate-blocked” or “3′-phosphate-protected” nucleotide refers to nucleotides in which the hydroxyl group at the 3′-position is blocked by the presence of a phosphate containing moiety. Examples of 3′-phosphate-blocked nucleotides in accordance with the invention arc nucleotidyl-3′-phosphate monoester/nucleotidyl-2′,3′-cyclic phosphate, nucicotidyl-2′-phosphate monoester and nucleotidyl-2′or 3′-alkylphosphate diester, and nucleotidyl-2′or 3′-pyrophosphate. Thiophosphate or other analogs of such compounds can also be used, provided that the substitution does not prevent dephosphorylation resulting in a free 3′-OH by a phosphatase.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999); Ferrero et al, Monatshefte fur Chemie, 131: 585-616 (2000); Taunton-Rigby et al, J. Org. Chem., 38(5): 977-985 (1973); Uemura et al, Tetrahedron Lett., 30(29): 3819-3820 (1989); Becker et al, J. Biol. Chem., 242(5): 936-950 (1967); Tsien, International patent publication WO1991/006678.

In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

—O-Z

wherein -Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula ═C(R″′)₂ wherein each R″′ may be the same or different and is selected from the group comprising hydrogen and halogen atoms and alkyl groups, with the proviso that in some embodiments the alkyl of each R″′ has from 1 to 3 carbon atoms; and wherein the molecule may be reacted to yield an intermediate in which each R″ is exchanged for H or, where Z is −(R′)₂—F, the F is exchanged for OH, SH or NH₂, preferably OH, which intermediate dissociates under aqueous conditions to afford a molecule with a free 3′-OH; with the proviso that where Z is −C(R′)₂—S—R″, both R′ groups are not H. In certain embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In certain embodiments, -Z of the modified nucleotide or nucleoside is of formula —C(R′)₂—N3. In certain embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, Mass.).

In a further embodiments, the 3′-blocked nucleotide triphosphate is blocked by either a 3′-O-azidomethyl, 3′-O—NH₂ or 3′-O-allyl group.

In still other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

In some embodiments, 3′-O— protection groups are electrochemically labile groups. That is, deprotection or cleavage of the protection group is accomplished by changing the electrochemical conditions in the vicinity of the protection group which result in cleavage. Such changes in electrochemical conditions may be brought about by changing or applying a physical quantity, such as a voltage difference or light to activate auxiliary species which, in turn, cause changes in the electrochemical conditions at the site of the protection group, such as an increase or decrease in pH. In some embodiments, electrochemically labile groups include, for example, pH-sensitive protection groups that are cleaved whenever the pH is changed to a predetermined value. In other embodiments, electrochemically labile groups include protecting groups which are cleaved directly whenever reducing or oxidizing conditions are changed, for example, by increasing or decreasing a voltage difference at the site of the protection group.

### Base Protection Groups

A wide variety of protection groups (or equivalently, “base protecting moieties”) may be employed to reduce or eliminate the formation of secondary structures in the course of polynucleotide chain extensions. Generally the conditions for removing base protection groups are orthogonal to conditions for removing 3′-O-blocking groups. In particular, where removal, or de-blocking, conditions for 3′-O-blocking groups are acidic, then base protection groups may be selected to be base labile. Under such circumstances, many base labile protection groups have been developed in phosphoramidite synthesis chemistry due to the use of acid labile 5′-O-trityl-protected monomers, e.g. Beaucage and Iyer, Tetrahedron Letters, 48(12): 2223-2311 (1992). In particular, acyl and amidine protecting groups for phosphoramidite chemistry are applicable in embodiments of the present invention (e.g. the protecting groups of Table 2 and Table 3 of Beaucage and Iyer (cited above)). In some embodiments, base protecting groups are amidines, such as described in Table 2 of Beaucage and Iyer (cited above). Generally, base-protected 3′-O-blocked nucleoside triphosphate monomers may be synthesized by routine modifications of methods described in the literature, such as described in the examples below.

In some embodiments, a base protecting group is attached to the 6-nitrogen of deoxyadenosine triphosphate, the 2-nitrogen of deoxyguanosine triphosphate, and/or the 4-nitrogen of deoxycytidine triphosphate. In some embodiments, a base protecting group is attached to all of the indicated nitrogens. In some embodiments, a base protecting group attached to a 6-nitrogen of deoxyadenosine triphosphate is selected from the group consisting of benzoyl, phthaloyl, phenoxyacetyl, and methoxyacetyl; a base protecting group attached to the 2-nitrogen of deoxyguanosine triphosphate is selected from the group consisting of isobutyryl, isobutyryloxyethylene, acetyl, 4-isopropyl-phenoxyacetyl, phenoxyacetyl, and methoxyacetyl; and a base protecting group attached to said 4-nitrogen of deoxycytidine triphosphate is selected from the group consisting of benzoyl, phthaloyl, acetyl, and isobutyryl.

In some embodiments, a protecting group attached to the 6-nitrogen of deoxyadenosine triphosphate is benzoyl; a base protecting group attached to the 2-nitrogen of deoxyguanosine triphosphate is isobutryl or dimethylformamidine; and the base protecting group attached to the 4-nitrogen of deoxycytidine triphosphate is acetyl.

In some embodiments, a base protecting group attached to the 6-nitrogen of deoxyadenosine triphosphate is phenoxyacetyl; a base protecting group attached to the 2-nitrogen of deoxyguanosine triphosphate is 4-isopropyl-phenoxyacetyl or dimethylformamidine; and the base protecting group attached to the 4-nitrogen of deoxycytidine triphosphate is acetyl.

In some embodiments, base protecting moieties are removed (i.e. the product is deprotected) and product is cleaved from a solid support in the same reaction. For example, an initiator may comprise a ribo-uridine which may be cleaved to release the polynucleotide product by treatment with 1 M KOH, or like reagent (ammonia, ammonium hydroxide, NaOH, or the like), which simultaneously removes base-labile base protecting moieties.

### Further Modifications of Elongation Conditions

In addition to providing 3′-O-blocked dNTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like.

### Base Analogs

In some embodiments, 3′-O-protected-nucleoside triphosphate monomers comprising base analogs may be employed for disrupting certain secondary structures, e.g. G-quadruplexes, which increase the likelihood of failure sequences occurring. In many cases, the presence of base analogs are acceptable in a synthesis product; that is, the presence of a base analog in a nucleotide of a primer may be acceptable in a polymerase chain reaction (PCR) assay. In some embodiments, in the presence of a tract of guanosine (G) in a polynucleotide to be synthesized, which may form a G-quadruplex structure, one or more of the G's in the tract may be substituted with deoxyinosine and/or 7-deaza-2′-deoxyguanosines to prevent the formation of G-quadruplexes during synthesis. In some embodiments, G's of a G tract in a polynucleotide are substituted with only 7-deaza-2′-deoxyguanosines. In some embodiments, if the number of G's substituted with 7-deaza-2′-deoxyguanosines depresses the melting temperature of a polynucleotide product to an unacceptable degree, a proportion of the analogs used in the substitutions may comprise 8-aza-7-deazaguanosine. G-quadruplex structure may be predicted using available algorithms, e.g. Lombardi et al, Nucleic Acids Research, 48(1): 1-15 (2020), and like references. Triphosphate monomers of the 3′-O-protected nucleoside analogs may be synthesize following techniques in the literature, e.g. cited above and Seela, U.S. Pat. No. 5,990,303. In some embodiments, a G tract is sequence segment of greater than 4 nucleotides in a polynucleotide containing more than 25% G's, or more than 30% G's, or more than 40% G's. In other embodiments, a G tract is a sequence segment conforming to the motif G₃₊N₁₋₇G₃₊N₁₋₇G₃₊N₁₋₇G₃₊, where “N” is any nucleotide and “3+” means 3 or more G's in a row. In some embodiments, the number of G's replaced by 7-deaza-guanosines may be from 1 to 100% of the G's in the tract, or from 1 to 50% of the G's in the tract, or from 1 to 25% of the G's in the tract, or from 1 to 10% of the G's in the tract. In some embodiments, such percentages of substitutions may be accomplished by selecting specific G's in a G tract for substitution, or such percentages substitutions may be accomplished statistically by using mixtures of G and 7-deaza-G in the addition step of one or more G's in the G tract of a polynucleotide being synthesized.

In some embodiments, the above methods for synthesizing a polynucleotide having a G tract may be implemented by the following steps: a) providing an initiator having a free 3′-hydroxyl; and b) repeating until the polynucleotide is synthesized cycles of (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′-hydroxyls, until the polynucleotide is formed, wherein in the G tract of the polynucleotide at least one G is substituted with an inosine or a 7-deazaguanosine. Whenever the polynucleotide is a polydeoxynucleotide, at least one G of the G tract is substituted with a deoxyinosine or a 7-deaza-2′-deoxyguanosine. In some embodiments, whenever the polynucleotide is a DNA, at least one G of the G tract is substituted with a 7-deaza-2′-deoxyguanosine.

### Base Protecting Moieties with Additional Functionalities

In some embodiments, base protecting moieties may be selected that include additional functionalities, such as, capture moieties, reporter groups, exonuclease blockers, or the like. Reporter groups may include fluorescent dyes, mass labels, electrochemical labels, or the like. In some embodiments, a base protecting moiety may include a capture moiety which may be used to separate or enrich full length polynucleotides from failure sequences. For example, in some embodiments, such base protecting moieties may be employed in a final cycle of dNTP addition, then after release or cleavage of the product from the synthesis support, the product is exposed under capture conditions to a support comprising a complement of the capture moiety (i.e. a capturing step is implemented), so that polynucleotides with a capture moiety may be separated from those without, thereby producing an enriched population of full length polynucleotide product. An optional washing step may be implemented, after which a cleavaging or deprotecting step may be implemented to release a product enriched in full length polynucleotides. As above, deprotecting or removing the protecting moieties with capture moieties results in a native polynucleotide product, that is, in a polynucleotide product having exocyclic amines without any unnatural adducts, or remnants of the protecting moiety.

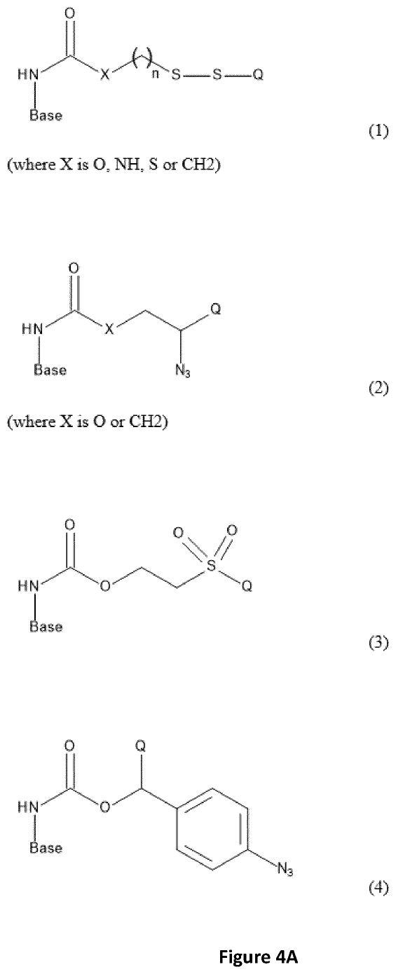

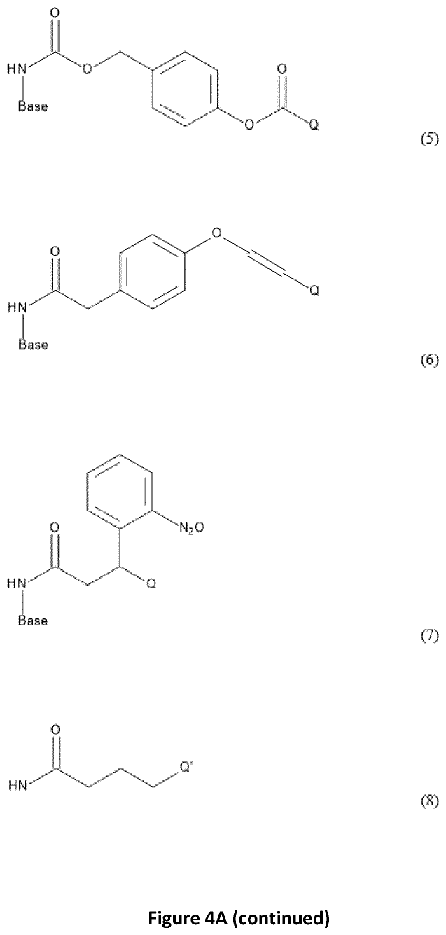

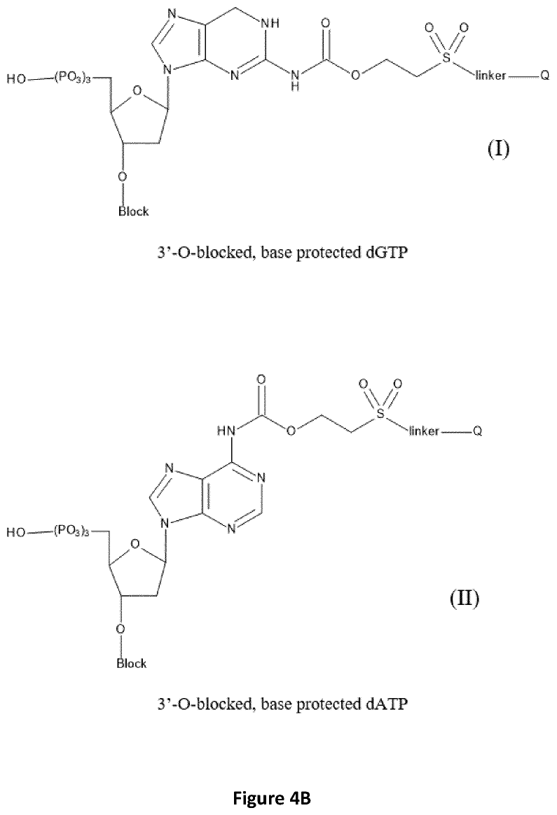

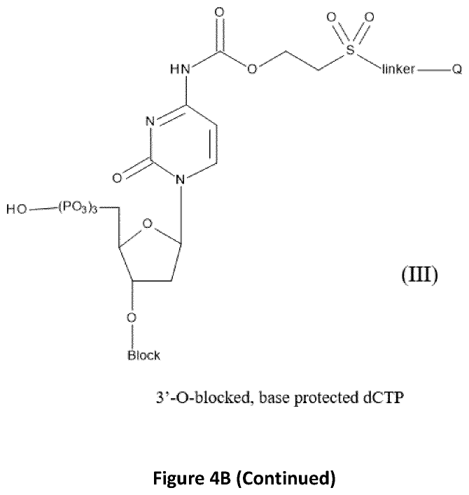

In some embodiments, such base protecting moieties are acyl protecting groups linked to a moiety carrying an additional functionality, designated as “Q” in FIG. 4A. Q may represent a capture moiety, such as a biotin, a reporter group, a nuclease blocker, or the like. In some embodiments, Q represents a capture moiety. A capture moiety may include groups that form covalent bonds in a capture step, such as aldol reactions, Diels-Alder reactions, Friedel-Crafts reactions, alkyne metathesis, cycloaddition, boronic acid condensation, and the like (e.g. reviewed in Jin et al, Chem. Soc. Rev., 42: 6634 (2013)), and groups that form noncovalent bonds in a capture step, such as, biotins that are captured by streptavidins, and fluoresceins, dinitrophenols, digoxigenins, or the like, that are captured by antibodies. FIG. 4A provides exemplary base protecting moieties that include capture moieties and Table I below gives information on their use with the invention.

TABLE 1

| Formula | Deprotection | |
|---|---|---|
| in FIG. 4A | Conditions | Reference |
| 1 | DTT or TCEP | Aissi et al, European Journal of Medicinal Chemistry, |
| | | 120: 304-312 (2016) |
| 2 | TCEP | |
| 3 | >pH 8 | Cowell et al, ChemBioChem Comm., 18: 1688-1691 |
| | | (2017) |
| 4 | Aryl phosphines | Lukasak et al, Scientific Reports, 9: 1470 (2019) |
| 5 | lipase | Sauerbrei et al, Angew. Chem. Int., 37(8): 1143-1146 |
| | | (1998) |
| 6 | tetrazine | Neumann et al, ChemBioChem, 18: 91-95 (2017) |
| 7 | light | Liang et al, Org. Lett., 18(5): 1174-1177 (2018) |
| 8 | NH3, NaOH | Beaucage et al, Tetrahedron, 48(12): 2223-2311 (1992) |
| | Penicillin-G-amidase | Amir et al, Chem. Comm., 14: 1614-1615 (2004) |
| — | light | Gisbert-Garzaran et al, Chem. Eng. J., 340: 24-31 |
| | | (2018) |

FIG. 4B illustrates exemplary 3′-O-blocked, base protected nucleoside triphosphates for use with methods of the invention. In the formulas of FIG. 4B, “linker” may be any suitable linker compatible with polymerase incorporation, such as, 1-4 carbon alkyl, or the like; Q may be biotin, desbiotin and biotin mimics, e.g. Liu et al, Chem. Soc. Rev., 46(9): 2391-2403 (2017); “base” is typically adenine, guanine or cytosine (wherein such base is part of a dNTP); and “block” may be as described above, but particularly, a cleavable organic moiety with or without heteroatoms having a molecular weight of 100 or less, or selected from the group consisting of methyl, 2-nitrobenzyl, allyl, amine, azidomethyl, tert-butoxy ethoxy, 2-cyanoethyl, and propargyl.

### Template-Free Polymerases

A variety of different template-free polymerases are available for use in methods of the invention. Template-free polymerases include, but are not limited to, polX family polymerases (including DNA polymerases β, λ and μ), poly(A) polymerases (PAPs), poly(U) polymerases (PUPs), DNA polymerase θ, and the like, for example, described in the following references: Ybert et al, International patent publication WO2017/216472; Champion et al, U.S. Pat. No. 10,435,676; Champion et al, International patent publication WO2020/099451; Yang et al, J. Biol. Chem., 269(16): 11859-11868 (1994); Motea et al, Biochim Biophys. Acta, 1804(5): 1151-1166 (2010). In particular, terminal deoxynucleotidyltransferases (TdTs) and its variants are useful in template-free DNA synthesis.

In some embodiments, enzymatic synthesis methods employ TdT variants that display increased incorporation activity with respect to 3′-O-modified nucleoside triphosphates. For example, such TdT variants may be produced using techniques described in Champion et al, U.S. Pat. No. 10,435,676, which is incorporated herein by reference. In some embodiments, a TdT variant is employed having an amino acid sequence at least 60 percent identical to a TdT having an amino acid sequence of any of SEQ ID NOs 2-31 and one or more of the substitutions listed in Table 1, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 1. In some embodiments, the above percent identity value is at least 80 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant. Thus, for example, if the reference sequence and the variant sequence each comprised 100 amino acids and the variant sequence had mutations at positions 25 and 81, then the percent homology would be in regard to sequences 1-24, 26-80 and 82-100.

In regard to (ii), such 3′-O-modified nucleotide may comprise a 3′-O—NH2-nucleoside triphosphate, a 3′-O-azidomethyl-nucleoside triphosphate, a 3′-O-allyl-nucleoside triphosphate, a 3′O-(2-nitrobenzyl)-nucleoside triphosphate, or a 3′-O-propargyl-nucleoside triphosphate.

TABLE 2

| SEQ ID | | | | | | |
|---|---|---|---|---|---|---|
| NO | Animal | Substitutions | | | | |
| 1 | Mouse | M192R/Q | C302G/R | R336L/N | R454P/N/A/V | E457N/L/T/S/K |
| 2 | Mouse | M63R/Q | C173G/R | R207L/N | R325P/N/A/V | E328N/L/T/S/K |
| 3 | Bovine | M63R/Q | C173G/R | R207L/N | R324P/N/A/V | E327N/L/T/S/K |
| 4 | Human | M63R/Q | C173G/R | R207L/N | R324P/N/A/V | E327N/L/T/S/K |
| 5 | Chicken | — | C172G/R | R206L/N | R320P/N/A/V | — |
| 6 | Possum | M63R/Q | C173G/R | R207L/N | R331P/N/A/V | E334N/L/T/S/K |
| 7 | Shrew | M63R/Q | C173G/R | R207L/N | — | E328N/L/T/S/K |
| 8 | Python | — | C174G/R | R208L/N | R331P/N/A/V | E334N/L/T/S/K |
| 9 | Canine | M73R/Q | C173G/R | R207L/N | R325P/N/A/V | E328N/L/T/S/K |
| 10 | Mole | M64R/Q | C174G/R | R208L/N | — | E329N/L/T/S/K |
| 11 | Pika | M61R/Q | C171G/R | R205L/N | R323P/N/A/V | E326N/L/T/S/K |
| 12 | Hedgehog | M63R/Q | C173G/R | R207L/N | R328P/N/A/V | E331N/L/T/S/K |
| 13 | Tree shrew | — | C173G/R | R207L/N | R325P/N/A/V | E328N/L/T/S/K |
| 14 | Platypus | M63R/Q | C182G/R | R216L/N | R338P/N/A/V | E341N/L/T/S/K |
| 15 | Jerboa | M66R/Q | C176G/R | R210L/N | R328P/N/A/V | E331N/L/T/S/K |
| 16 | Canary | — | C170G/R | R204L/N | R326P/N/A/V | E329N/L/T/S/K |
| 17 | Neopelma | — | C158G/R | R192L/N | R314P/N/A/V | E317N/L/T/S/K |
| 18 | Alligator | — | — | R205L/N | R327P/N/A/V | E330N/L/T/S/K |
| 19 | Xenopus | — | — | R205L/N | R324P/N/A/V | E327N/L/T/S/K |
| 20 | Tiger snake | — | — | R205L/N | R327P/N/A/V | E330N/L/T/S/K |
| 21 | Brown trout | — | — | R192L/N | R311P/N/A/V | E314N/L/T/S/K |
| 22 | Electric eel | — | — | R205L/N | R321P/N/A/V | E325N/L/T/S/K |
| 23 | Walking fish | — | — | R205L/N | R322P/N/A/V | E325N/L/T/S/K |
| 24 | Guppy | — | — | R205L/N | R322P/N/A/V | E325N/L/T/S/K |
| 25 | Rat | M48R/Q | C158G/R | R192L/N | R310P/N/A/V | E313N/L/T/S/K |
| 26 | Rat | M61R/Q | C171G/R | R205L/N | R323P/N/A/V | E326N/L/T/S/K |
| 27 | Colobus monkey | M61R/Q | C171G/R | R205L/N | R323P/N/A/V | E326N/L/T/S/K |
| 28 | Pig | M61R/Q | C171G/R | R205L/N | R323P/N/A/V | E326N/L/T/S/K |
| 29 | Tiger | M61R/Q | C171G/R | R205L/N | R323P/N/A/V | E326N/L/T/S/K |
| 30 | Water buffalo | M48R/Q | C158G/R | R192L/N | R310P/N/A/V | E313N/L/T/S/K |
| 31 | Marmot | M61R/Q | C171G/R | R205L/N | R323P/N/A/V | E326N/L/T/S/K |

In some embodiments, further TdT variants for use with methods of the invention include one or more of the substitutions of methionine, cysteine, arginine (first position), arginine (second position) or glutamic acid, as shown in Table 2.

TdT variants of the invention as described above each comprise an amino acid sequence having a percent sequence identity with a specified SEQ ID NO, subject to the presence of indicated substitutions. In some embodiments, the number and type of sequence differences between a TdT variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 3A.

TABLE 3A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 3B.

TABLE 3B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, He |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

### Kits

Kits for carrying out methods of the invention may comprise 3′-O-protected-nucleoside triphosphate monomers that comprise a base having amidine- or acyl-protected exocyclic amines or base analogs (which also may have amidine or acyl-protected exocyclic amines). In some embodiments, a 3′-O-protected dNTP monomer having a base analog of a kit comprises a 3′-O-protected-2′-deoxy-7-deazaguanosine triphosphate.

### EXAMPLE 1

### Amidine and Acyl Protection of Exocyclic Amines of 3′-O-Amino-Protected-2′-Deoxynucleoside Triphosphates

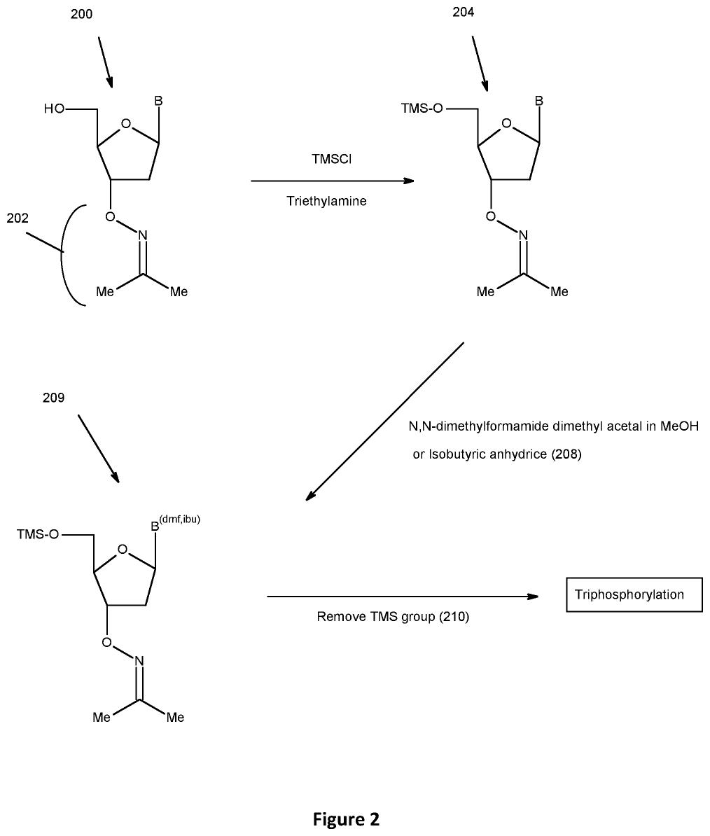

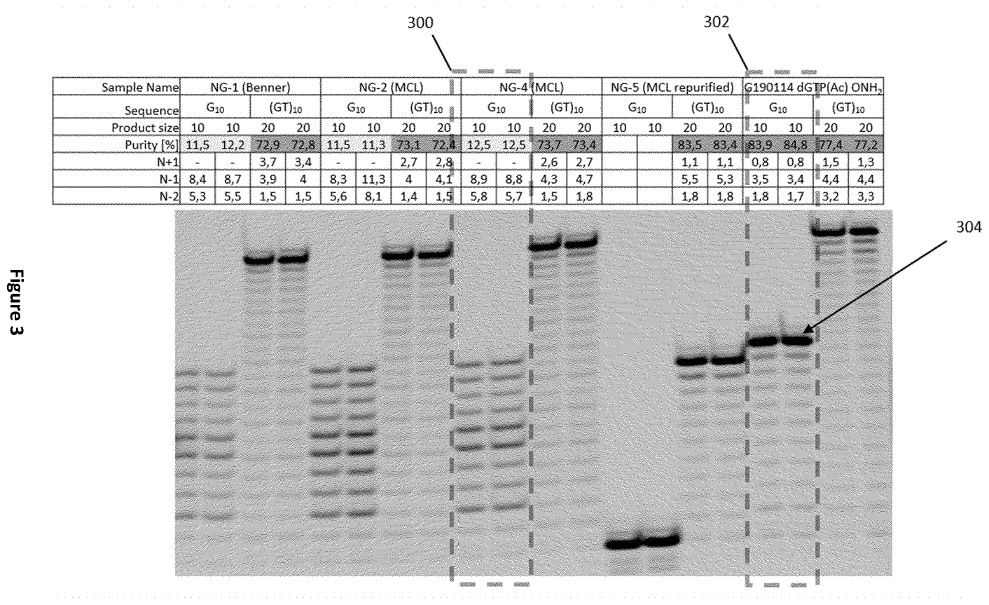

Amidine and acyl protection groups may be attached to 3′-O-amino-protected-2′-deoxynucleoside triphosphates using the scheme of FIG. 2. Compound (200) with 3′-O-oxime moiety is obtained as described in Benner, U.S. Pat. No. 8,212,020 which is incorporated herein by reference (e.g. see compound 3e in Benner). Here “B” represents adenine, guanine or cytosine. 5′-hydroxyl of compound (200) is protected with a trimethylsilyl group using a conventional procedure, e.g. Ti et al, J. Amer. Chem. Soc., 104: 1316-1319 (1982); Kierzek, Nucleosides and Nucleotides, 4: 641-649 (1985), to give compound (204). Whenever B is guanine or adenine, compound (204) is combined (208) with N,N-dimethylformamide dimethyl acetal in methanol as taught by Vu et al, Tetrahedron Letters, 31(50): 7269-7272 (1990) to give compound (209) with 5′-TMS-O-3′-O—(N-acetone-oxime)-dG^{dmf} and 5′-TMS-O-3′-O—(N-acetone-oxime)-dA^{dmf}. Whenever B is cytosine, compound (204) is combined with isobutyric anhydride as taught by Vu et al (cited above) to give 5′-TMS-O-3′-O—(N-acetone-oxime)-dC^{ibu}. After removal of the TMS protecting group (210) (e.g. treatment with tetrabutylammonium fluoride), the resulting compounds may be triphosphorylated and the 3′-O—N-acetone-oxime groups converted to amines as taught by Benner.

### EXAMPLE 2

### Increased Yield of (dG)₁₀ Using 3′-O-Amino-Protected-N2-Acetyl-2′-Deoxyguanosine Triphosphates

In this example, yields of (dG)₁₀ were compared after template-free enzymatic synthesis using dGTP monomers with unprotected bases and dGTP monomers with acecylated N2 nitrogens. (dG)10 oligonucleotides otherwise were synthesized as described above. Results are shown in the electropherograms of FIG. 3. Electropherogram ladders (300) show separated product after synthesis using non-base protected 3′-O—NH2-2′-deoxyguanosine triphosphate and electropherogram ladders (302) show separated product after synthesis using 3′-O—NH2-N2-acetyl-2′-deoxyguanosine triphosphate. The dominant bands (304) of high molecular weight product in ladders (302) show that more full-length product was produced using base-protected monomers.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, N.Y., 1992); Lehninger, Biochemistry, Second Edition (Worth Publishers, N.Y., 1975); Strachan and Read, Human Molecular Genetics, Second Edition (Wiley-Liss, N.Y., 1999).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to inefficiency motifs that are conserved among the amino acid sequences of TdTs of evolutionarily related species, e.g. genus, families, or the like. Examples of such conserved inefficiency motifs are described in Motea et al, Biochim Biophys. Acta. 1804(5): 1151-1166 (2010); Delarue et al, EMBO J., 21: 427-439 (2002); and like references.

“Kit” refers to any delivery system, such as a package, for delivering materials or reagents for carrying out a method implemented by a system or apparatus of the invention. In some embodiments, consumables materials or reagents are delivered to a user of a system or apparatus of the invention in a package referred to herein as a “kit.” In the context of systems and apparatus of the invention, such delivery systems include, usually packaging methods and materials that allow for the storage, transport, or delivery of materials, such as, 3′-O-protected-dNTPs. For example, kits may include one or more enclosures (e.g., boxes) containing the 3′-O-protected-dNTPs and/or supporting materials. Such contents may be delivered to the intended recipient together or separately. For example, a first container may contain a 3′-O-protected-dNTP with exocyclic nitrogens having protection groups, while a second or more containers contain a 3′-O-protected-deoxyguanosine triphosphate, a template-free polymerase, for example, a specific TdT, and appropriate buffers.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, N.Y., 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, N.Y., 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, N.Y., 2003).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/ or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine) Preferably, the term “substitution” refers to the replacement of an amino acid residue by another selected from the naturally-occurring standard 20 amino acid residues. The sign “+” indicates a combination of substitutions. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr). In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1. A method of synthesizing a polynucleotide having a predetermined sequence, the method comprising the steps of:
a) providing an initiator having a free 3′-hydroxyl; and
b) repeating until the polynucleotide is synthesized cycles of (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′-hydroxyls, until the polynucleotide is formed, wherein the elongation conditions are selected to prevent hydrogen bonding or base stacking.

2. The method of claim 1, wherein said elongation conditions provide that at least one 3′-O-blocked nucleoside triphosphate has a base protecting moiety attached to its base to prevent hydrogen bonding.

3. The method of claim 2, wherein said 3′-O-blocked nucleoside triphosphate has a base protecting moiety attached to a nitrogen or to an oxygen of its base.

4. The method of claim 3, wherein said 3′-O-blocked nucleoside triphosphate has said base protecting moiety attached to a nitrogen.

5. The method of claim 4, wherein said nitrogen of said base of said 3′-O-blocked nucleoside triphosphate is an exocyclic nitrogen.

6. The method of claim 5, wherein said base protecting moiety is attached to 6-nitrogen of deoxyadenosine triphosphate, 2-nitrogen of deoxyguanosine triphosphate, or 4-nitrogen of deoxycytidine triphosphate.

7. The method of claim 6, wherein said base protecting moiety is an acyl protecting group.

8. The method of claim 6, wherein said base protecting moiety attached to said 6-nitrogen of deoxyadenosine triphosphate is selected from the group consisting of benzoyl, phthaloyl, phenoxy acetyl, and methoxy acetyl; wherein said base protecting moiety attached to said 2-nitrogen of deoxyguanosine triphosphate is selected from the group consisting of isobutyryl, isobutyryloxyethylene, acetyl, 4-isopropyl-phenoxyacetyl, phenoxyacetyl, and methoxyacetyl; and
wherein said base protecting moiety attached to said 4-nitrogen of deoxycytidine triphosphate is selected from the group consisting of benzoyl, phthaloyl, acetyl, and isobutyryl.

9. The method of claim 6, wherein said base protecting moiety attached to said 6-nitrogen of deoxyadenosine triphosphate is benzoyl or dimethylformamidine; wherein said base protecting moiety attached to said 2-nitrogen of deoxyguanosine triphosphate is acetyl or dimethylformamidine; and wherein said base protecting moiety attached to said 4-nitrogen of deoxycytidine triphosphate is acetyl.

10. The method of claim 6, wherein said base protecting moiety attached to said 6-nitrogen of deoxyadenosine triphosphate is dimethylformamidine; wherein said base protecting moiety attached to said 2-nitrogen of deoxyguanosine triphosphate is dimethylformamidine; and wherein said base protecting moiety attached to said 4-nitrogen of deoxycytidine triphosphate is acetyl.

11. The method of claim 2, wherein said base protecting moiety is base labile.

12. The method of claim 2, wherein said base protecting moiety is an amidine.

13. The method of claim 2, wherein said method includes removing said base protecting moieties from nucleotides of the polynucleotide.

14. The method of any of claim 1, wherein said initiator is attached to a solid support.

15. The method of claim 2, wherein said initiator comprises a base-cleavable nucleoside and said base protecting moieties are base labile and wherein said step of removing comprises treating said polynucleotide with base so that base protecting moieties and the base-cleavable nucleoside are cleaved in the same reaction.

16. The method of claim 1, wherein said elongation conditions include a denaturation agent.

17. The method of claim 16, wherein said denaturation agent is selected from the group consisting of water miscible solvents having a dielectic constant less than that of water and chaotropic agents.

18. The method of claim 16, wherein said denaturation agent is selected from the group consisting of formamide, guanidine, sodium salicylate, dimethyl sulfoxide (DMSO), propylene glycol, and urea.

19. The method of claim 2, wherein said 3′-O-protecting group is selected from the group consisting of 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

20. The method of claim 19 wherein said 3′-O-protecting group is azidomethyl.

21. The method of claim 19 wherein said 3′-O-protecting group is amine.

22. A method of synthesizing a polynucleotide having a predetermined sequence, the method comprising the steps of:
a) providing an initiator having a free 3′-hydroxyl;
b) repeating until the polynucleotide is synthesized cycles of (i) contacting under elongation conditions the initiator or elongated fragments having free 3′-O-hydroxyls with a 3′-O-blocked nucleoside triphosphate and a template-independent DNA polymerase so that the initiator or elongated fragments are elongated by incorporation of a 3′-O-blocked, base protected nucleoside triphosphate to form 3′-O-blocked elongated fragments, and (ii) deblocking the elongated fragments to form elongated fragments having free 3′-hydroxyls, until the polynucleotide is formed, wherein the elongation conditions are selected to prevent hydrogen bonding or base stacking; wherein a final cycle comprises only step (i) and wherein the 3′-O-blocked, base protected nucleoside triphosphate comprises a base protecting moiety comprising a capture moiety; and
c) capturing the polynucleotide with a complement of the capture moiety.

23. The method of claim 22 further comprising a step of deblocking said captured polynucleotide.


SEQUENCE LISTING (31 sequences)

SEQ ID NO: 1 - protein, 510 aa, ARTIFICIAL SEQUENCE
  TdT full length mouse
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 381 aa, ARTIFICIAL SEQUENCE
  TdT truncated mouse sequence
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 3 - protein, 380 aa, ARTIFICIAL SEQUENCE
  Bovine truncated (catalytic domain)
DYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 4 - protein, 380 aa, ARTIFICIAL SEQUENCE
  TdT Human truncated
DYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 5 - protein, 376 aa, ARTIFICIAL SEQUENCE
  TdT Chicken 1 truncated
QYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 6 - protein, 387 aa, ARTIFICIAL SEQUENCE
  Possum truncated
SANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 7 - protein, 381 aa, ARTIFICIAL SEQUENCE
  truncated shrew
DCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 8 - protein, 387 aa, ARTIFICIAL SEQUENCE
  Python truncated
EKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 9 - protein, 381 aa, ARTIFICIAL SEQUENCE
  truncated dog
DYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 10 - protein, 382 aa, ARTIFICIAL SEQUENCE
  TRUNC MOLE
GDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 11 - protein, 379 aa, ARTIFICIAL SEQUENCE
  Pika trunk
EYSANPSPGPQATPAVYKISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 12 - protein, 384 aa, ARTIFICIAL SEQUENCE
  TRUNC HEDGEHOG
DASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 13 - protein, 381 aa, ARTIFICIAL SEQUENCE
  truncated tree shrew
DHSTSPSPGPQKTPALAVQKISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 14 - protein, 394 aa, ARTIFICIAL SEQUENCE
  TRUNCATED PLATYPUS
LTNSAPINCMTETPSLATKQVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 15 - protein, 384 aa, ARTIFICIAL SEQUENCE
  TRUNCATED JERBOA
SSELELLDVSWLIECMGAGKPVEMTGRHQLVKQTFCLPGFILQDAFDILAENCEFRENEASCVEFMRAASVLKSLPFPIISVKDTEGIPWLGGKVKCVIEEIIEDGESSEVKALLNDERYKSFKLFTSVFGVGLKTAERWFRMGFRTLSTVKLDKSLTFTRMQKAGFLHYEDLVSCVTRAEAEAVSVLVQQAVVAFLPDALVSMTGGFRRGKKIGHDVDFLITSPEATEEEEQQLLHKVTNFWEQKGLLLYCDHVESTFEKCKLPSRKVDALDHFQKCFLILKLYRERVDSVKSSQQEGKGWKAIRVDLVMCPYECRAFALLGWTGSRQFERDLRRYATHERKMRLDNHALYDKTKRVFLKAESEEEIFAHLGLEYIEPLERNA

SEQ ID NO: 16 - protein, 382 aa, ARTIFICIAL SEQUENCE
  trunc wt Serinus canaria (canary)
SPPLNTPELEMPSFIATKVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 17 - protein, 370 aa, ARTIFICIAL SEQUENCE
  trunc wt Neopelma chrysocephalum (saffron- crested neopelma)
FIARKVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 18 - protein, 383 aa, ARTIFICIAL SEQUENCE
  trunc wt  Alligator sinensis
SPSPVPGSQNVPAPSVDKVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 19 - protein, 380 aa, ARTIFICIAL SEQUENCE
  trunc wt  Xenopus laevis
SPSPVPGSQNVPAPSEVKVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 20 - protein, 383 aa, ARTIFICIAL SEQUENCE
  trunc wt (Notechis scutatus) tiger snake
SPSPVPGSQNVPAPSVDSISPYACQRRTTLKNYNQKFTDAFEILVENYEFNENKEFCLAFGRAASLLKCLPFTVVRVNDIEGLPWMGKQVREIIEDILEEGESSKVKAVLNDENYRSIKLFTSVFGVGLRTSEKWYRMGLRTLEEVKCNKNLTLTRMQKAGFFYYDDLISSVSKAEADAATQIVQDTVWKILPNAVVTLTGGFRRGKQTGHDVDFLITVPGSRQEEELLHPVIDIWKKQELLLYYDLIESTFENTKLPSRKVDALDHFQKCFAILKVHKERVNKGSAVQSNVFAEEGTKDWKAIRVDLVVTPFQHYAFALLGWTGSRQFERDLRRYATQEKKMMLDNHALYDKTKKIFLSAANEEEIFSHLGLDYLEPWERNA

SEQ ID NO: 21 - protein, 367 aa, ARTIFICIAL SEQUENCE
  trunc wt (Salmo trutta) brown trout
TAAANVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 22 - protein, 377 aa, ARTIFICIAL SEQUENCE
  trunc wt (Electrophorus electricus) electric eel
SPSPVPGSQNVPAPSLPTVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 23 - protein, 378 aa, Anabas testudineus
SPSPVPGSQNVPAPSVATVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 24 - protein, 378 aa, ARTIFICIAL SEQUENCE
  trunc wt (Poecilia reticulate) guppy
SPSPVPGSQNVPAPSVDKVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 25 - protein, 366 aa, ARTIFICIAL SEQUENCE
  trunc wt  short Rattus norvegicus
PLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 26 - protein, 379 aa, ARTIFICIAL SEQUENCE
  trunk wt  Long Rattus norvegicus
SLSPVPGSQTVPPPLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 27 - protein, 379 aa, ARTIFICIAL SEQUENCE
  trunc wt (Piliocolobus tephrosceles) monkey
SDSTNPGLPKTPPTAIQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 28 - protein, 379 aa, ARTIFICIAL SEQUENCE
  N27 trunc wt (Sus scrofa) pig
SASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 29 - protein, 379 aa, ARTIFICIAL SEQUENCE
  trunc wt (Panthera tigris altaica) Siberian tiger
YSASPNPELQKTPPLVKKIPLYACQRRTTLNNFNHVFTDAFEVLAENYEFKENEVSSATFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITSPGSTDEEEEELLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 30 - protein, 365 aa, ARTIFICIAL SEQUENCE
  trunc wt (Bubalus bubalis) water buffalo
LAVKKISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 31 - protein, 379 aa, ARTIFICIAL SEQUENCE
  N93or trunc wt (Marmota flaviventris) marmot
PDHSNSDPQRIPPPAVQTISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA